(19) United States
(12) Patent Application Publication
(10) Pub. No.: US 2021/0022705 A1
(43) Pub. Date: Jan. 28, 2021

(54) ULTRASONIC CT APPARATUS, CONTAINER FOR ULTRASONIC CT APPARATUS, AND BREAST IMAGING METHOD

(71) Applicant: Hitachi, Ltd., Tokyo (JP)

(72) Inventors: Atsuro SUZUKI, Tokyo (JP); Yushi TSUBOTA, Tokyo (JP); Takahide TERADA, Tokyo (JP); Kenichi KAWABATA, Tokyo (JP); Kazuhiro YAMANAKA, Tokyo (JP); Wenjing WU, Tokyo (JP)

(21) Appl. No.: 16/816,722

(22) Filed: Mar. 12, 2020

(30) Foreign Application Priority Data
Jul. 26, 2019 (JP) 2019-138176

(51) Int. Cl. A61B 8/00; A61B 8/08; A61B 8/15

(52) U.S. Cl. CPC A61B 8/406; A61B 8/403; A61B 8/0825; A61B 8/5207; A61B 8/15; A61B 8/4488

(57) ABSTRACT

To provide an ultrasonic CT apparatus that can change an inclination of a side surface of a breast such that an ultrasonic wave is incident on a surface of an entire circumference of the breast at an angle close to perpendicular, and that has less psychological burden on a subject. A container provided with an opening to which the breast is inserted is disposed below a bed provided with a through hole to which the breast of the subject is inserted. In the container, a member that propagates or transmits the ultrasonic wave is disposed, and the member is relatively pressed against a side surface or a nipple portion of the breast such that an inclination of the side surface of the breast is close to perpendicular. A transducer array emits the ultrasonic wave around the breast with the inclination of the side surface being close to perpendicular.

### CROSS-REFERENCE TO RELATED APPLICATION

The present application claims priority from Japanese application JP 2019-138176, filed on Jul. 26, 2019, the contents of which is hereby incorporated by reference into this application.

### TECHNICAL FIELD

The present invention relates to a breast shaping method in an ultrasonic CT apparatus.

### BACKGROUND ART

PTL 1 discloses a breast ultrasonic computed tomography (CT) apparatus as a medical diagnostic apparatus in which an ultrasonic measurement is applied to detection of a breast cancer. In the breast ultrasonic CT apparatus, a ring-shaped transducer array, which is an ultrasonic transmitter and receiver, is disposed around a breast inserted into water, and emits ultrasonic waves to the breast from 360° in entire circumferential directions, and reflected signals and transmitted signals from the breast are measured for reconstruction of an image. Accordingly, a tomographic image of the breast is acquired. Information relating to a structure of a breast tissue is obtained from the reflected signals, and information relating to a sound speed and an attenuation of the ultrasonic waves for the tissue is obtained from the transmitted signals. Generally, the sound speed and an attenuation amount of the ultrasonic waves for a tumor are higher than those for normal tissues of surrounding mammary glands, fat, and the like. Therefore, it is possible to quantitatively detect the tumor from a tomographic image (transmitted wave image) of the sound speed or the attenuation amount of the ultrasonic waves.

In the breast ultrasonic CT apparatus, since the transducer array does not touch the breast and emits ultrasonic waves through water around the breast, when it is assumed that a shape of the breast is approximated to a cone and that an ultrasonic wave is emitted horizontally (parallel to a bottom surface of the cone) , the ultrasonic wave is not incident perpendicular to a surface of the breast. Because a sound speed of water around the breast is different from a sound speed of a breast skin, when the ultrasonic wave is not incident perpendicular to the surface of the breast, the ultrasonic waves are refracted and bent in a z direction (a direction perpendicular to a plane) from a plane where the transducer array exists (a plane parallel to the bottom surface of the cone). As a result, a rate at which an ultrasonic wave reflected in the breast or an ultrasonic wave transmitted through the breast reaches the transducer array is reduced, and therefore, a signal intensity output by the transducer array is reduced, resulting in degradation of an image quality. Therefore, it is desirable to shape the breast so that the ultrasonic wave is incident on the surface of the breast as perpendicularly as possible, or an incident angle (an angle formed with a normal line of the surface of the breast) is reduced.

PTL 2 discloses a breast image diagnostic apparatus using photoacoustic. This apparatus emits a laser to the breast in a direction from a nipple to a chest wall, and an acoustic signal generated from the breast is measured by a transducer array disposed around the breast to detect the tumor. At this time, in the technique of PTL 2, the breast is compressed and shaped by pushing the breast from the nipple to a chest wall direction with a balloon. Since this compression can reduce thickness of the breast, it is possible to reduce an attenuation of light of the laser in the breast and to make the light incident on an entire area of the breast.

On the other hand, PTL 3 proposes a shaping method in which the breast is stretched by sucking a nipple portion of the breast from below and pulling the nipple portion downward in order to reduce the incident angle of the ultrasonic wave to the surface of the breast in a breast ultrasonic CT apparatus.

### CITATION LIST

### Patent Literature

PTL 1: US Patent Application Publication 2018/0140273 specification

PTL 2: US Patent Application Publication 2016/0262628 specification

PTL 3: US Patent Application Publication 2017/0224305 specification

### SUMMARY OF INVENTION

### Technical Problem

In the photoacoustic technique of PTL 2, it is described that the breast is compressed by pushing the breast in the direction from the nipple to the chest wall direction with the balloon, but in the photoacoustic technique, the surface of the breast is emitted with light, and the ultrasonic waves are not incident from a side surface of the breast, and therefore, a shape of the side surface of the breast is not considered.

On the other hand, in a breast shaping method of PTL 3, since the nipple portion is sucked from below and pulled downward, a psychological burden is imposed on a patient when the nipple portion is pulled. In addition, it is necessary to add a mechanism for sucking the breast to an apparatus configuration, which leads to an increase in apparatus cost.

An object of the invention is to provide an ultrasonic CT apparatus that can change an inclination of a side surface of abreast such that ultrasonic waves is incident on a surface of an entire circumference of the breast at an angle close to perpendicular, and that has less psychological burden on a subject.

### Solution to Problem

In order to achieve the above object, according to the invention, an ultrasonic CT apparatus is provided, the ultrasonic CT apparatus including: a bed provided with a through hole to which a breast of a subject is inserted; a container disposed below the through hole and provided with an opening to which the breast is inserted; and a transducer array configured to emit an ultrasonic wave around the breast in the container, and to receive an ultrasonic wave from the breast. Here, in the container, a member that propagates or transmits the ultrasonic wave is disposed, and the member is configured to be relatively pressed against a side surface or a nipple portion of the breast such that an inclination of the side surface of the breast is close to perpendicular.

### Advantageous Effect

According to the invention, since the inclination of the side surface of the breast can be changed by pressing the member against the side surface or a bottom surface of the breast, it is possible to make the ultrasonic wave incident on a surface of an entire circumference of the breast from an angle close to perpendicular, to improve an image quality of a reflection image and a transmitted wave image, and to reduce a psychological burden on the subject.

### BRIEF DESCRIPTION OF DRAWINGS

FIG. 1 is a block diagram showing a configuration of a breast ultrasonic CT apparatus according to a first embodiment.

FIG. 2 is a perspective view showing a state in which a transducer array 3 is disposed around a breast in water.

FIG. 3 is a diagram showing a case where an ultrasonic wave is not incident perpendicular to a surface of the breast.

FIG. 4 is a top view of a case where a member 20 is disposed on a side surface of a tank 4.

FIGS. 5A and 5B are cross-sectional views showing a structure in which the member 20 is disposed on the side surface of the tank 4.

FIG. 6 is a cross-sectional view showing a structure in which the member 20 contained in a bag 21 is fixed to a surface of a bed 2 according to a first modification of the first embodiment.

FIG. 7 is a cross-sectional view showing a structure in which thickness of the member 20 changes in a z direction according to a second modification of the first embodiment.

FIGS. 8A and 8B are diagrams showing a state in which water is supplied to and drained from the member 20 in which water 23 is in a bag 22 according to a third modification of the first embodiment.

FIGS. 9A and 9B are diagrams showing how the thickness of the member 20 is adjusted by adjusting a water amount in the member 20 in which the water 23 is in the bag 22 according to a fourth modification of the first embodiment.

FIGS. 10A and 10B are cross-sectional views showing a state where a subject 1 inserts a breast 1a from a state where an upper end of the member 20 protrudes from the surface of the bed 2 according to a fifth modification of the first embodiment.

FIG. 11A is a block diagram showing a structure in which a camera 31 is disposed at a bottom portion of the tank 4 according to a sixth modification of the first embodiment, and FIG. 11B is a diagram showing an image imaged by the camera 31.

FIGS. 12A and 12B are block diagrams showing a structure for moving the tank 4 according to a seventh modification of the first embodiment.

FIGS. 13A and 13B are block diagrams showing a structure for moving the bed 2 according to an eighth modification of the first embodiment.

FIGS. 14A and 14B are top views showing a structure for moving the member 20 within the tank 4 according to a ninth modification of the first embodiment.

FIGS. 15A to 15C are block diagrams showing a structure for moving the member 20 in the tank 4 as seen from the side surface.

FIGS. 16A and 16B are top views showing a structure for moving a film member 20 within the tank 4 according to a tenth modification of the first embodiment.

FIGS. 17A and 17B are block diagrams showing a structure for moving the film member 20 in the tank 4 as seen from the side surface.

FIG. 18A is a top view showing a structure in which a plurality of members 20 are disposed in the tank 4 according to an eleventh modification of the first embodiment, and FIG. 18B is a diagram showing a state where the member 20 is pressed against a left breast 1a from a diagonal direction.

FIG. 19 is a top view showing a structure in which the member 20 is disposed in a half-circumferential area of an inner wall of the tank 4 according to a twelfth modification of the first embodiment.

FIG. 20 is a top view showing a structure in which the member 20 is disposed on an entire circumference of the inner wall of the tank.

FIGS. 21A and 21B are side views showing a structure for pushing up a member 51 using a member 52 having buoyancy according to a second embodiment.

FIG. 22 is a top view of a structure in which the member 51 is pushed up using the member 52 having buoyancy.

FIG. 23A is a block diagram showing a structure for detecting water overflowing from the tank 4 with a flow rate sensor 55, and FIG. 23B is a flowchart showing an operation of a control unit 7 that controls water injection using an output of the flow rate sensor 55.

FIG. 24 is a side view showing a structure for confirming water overflowing from the tank 4 with a camera 31 according to a first modification of the second embodiment.

FIG. 25 is a top view of a structure of the tank 4 and members 51 and 52 of FIG. 24.

FIG. 26 is a diagram showing a case where the member 51 has a recess according to a second modification of the second embodiment.

FIG. 27 is a cross-sectional view showing an example in which an auxiliary member 57 is attached to an end portion of the member 52 according to a third modification of the second embodiment.

FIGS. 28A and 28B are block diagrams showing a structure provided with an up-down movement mechanism 62 with a motor according to a fourth modification of the second embodiment.

FIG. 29 is a block diagram showing a structure provided with a mechanism for pushing up the breast from below with air according to a fifth modification of the second embodiment.

FIG. 30 is a block diagram showing a structure in which a member is lifted by a string and the breast is pushed up from below according to a sixth modification of the second embodiment.

FIG. 31 is a cross-sectional view of a structure of a third embodiment, the structure being a combination of the structures of the first and second embodiments.

FIG. 32 is a top view of a structure using the member 52 having buoyancy in which the member 20 is disposed on the side surface of the tank 4 according to the third embodiment.

FIG. 33 is a top view of a structure using the member 52 having buoyancy in which a plurality of members 20 are disposed on the side surface of the tank.

### DESCRIPTION OF EMBODIMENTS

An ultrasonic CT apparatus according to an embodiment of the invention will be described below with reference to the drawings.

In the ultrasonic CT apparatus according to the present embodiment, in a container provided with an opening to which a breast is inserted, a member that propagates or transmits an ultrasonic wave is disposed, and the member is relatively pressed against a side surface or a nipple of the breast such that an inclination of the side surface of the breast is close to perpendicular with respect to a chest wall.

Accordingly, since the inclination of the side surface of the breast can be changed by pressing the member against the side surface or a bottom surface of the breast, it is possible to make the ultrasonic wave incident on a surface of an entire circumference of the breast from an angle close to perpendicular, and to improve an image quality of a reflection image and a transmitted wave image acquired by the ultrasonic CT apparatus. In addition, a method of changing the inclination by pressing the member against the side surface or the bottom surface of the breast has a merit that a psychological burden on a subject is less than when the breast is pulled.

Hereinafter, specific embodiments will be described.

### First Embodiment

In the first embodiment, the ultrasonic CT apparatus is described in which the inclination of the side surface of the breast is changed by pressing the member against the side surface of the breast. First, a configuration of the ultrasonic CT apparatus will be described.

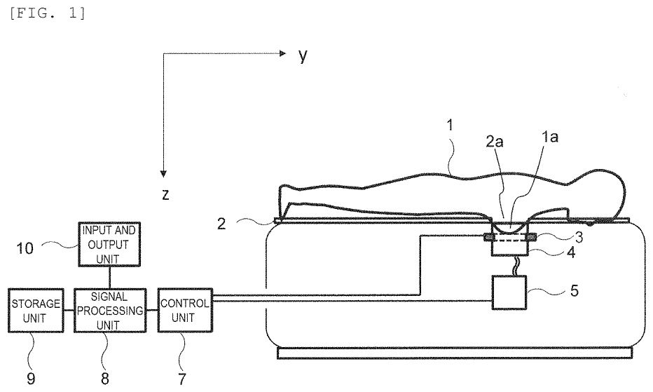

As shown in FIG. 1, the ultrasonic CT apparatus according to the first embodiment includes a bed 2 provided with a through hole 2a to which a breast 1a of a subject 1 is inserted, a container (hereinafter referred to as a tank) 4 disposed below the opening 2a and provided with an opening to which the breast 1a is inserted, and a transducer array 3 configured to emit an ultrasonic wave around the breast 1a in the tank 4 and receive an ultrasonic wave from the breast.

On the bed 2, the subject 1 is mounted facing down. The transducer array 3 is, for example, a ring-shaped array, and transmits and receives ultrasonic waves. The tank 4 is filled with water, and is connected to a reserve tank 5 for supplying water to the tank 4. The transducer array 3 and the reserve tank 5 are connected to a control unit 7, and the control unit 7 controls data collection by transmission and reception of ultrasonic waves by the transducer array 3, and adjusts a temperature of water in the reserve tank 5. A signal processing unit 8 is connected to the control unit 7, and converts the collected data into an image. The signal processing unit 8 is connected to a storage unit 9 that stores the collected data and the image, and to an input and output unit 10 that inputs a command and outputs an image.

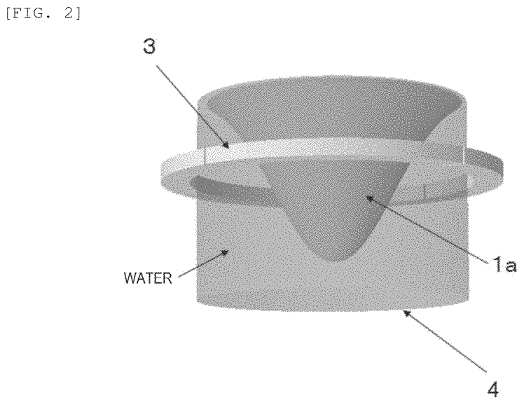

As shown in FIG. 2, the transducer array 3 has a ring shape, and is disposed outside the tank 4 in the present embodiment. From the transducer array 3, ultrasonic waves having a frequency of about several MHz are transmitted. Accordingly, an ultrasonic wave is emitted to the breast 1a which is the subject in water from 360° in entire circumferential directions. A part of the ultrasonic wave incident on the breast 1a is reflected on a surface of the breast 1a or a surface of a structure in the breast 1a, and these reflected waves are received by the transducer array 3. A part of the ultrasonic waves transmits through the breast 1a while being repeatedly refracted on the surface or an inside of the breast 1a, and these transmitted waves are also received by the transducer array 3.

Since a series of transmission and reception of these ultrasonic waves are performed from the entire circumference of the breast 1a as described above, the signal processing unit 8 reconstructs an image with received signals of the reflected wave, thereby acquiring a tomographic image representing a boundary of the structure. On the other hand, when the signal processing unit 8 reconstructs an image with received signals of the transmitted wave, two types of transmitted wave images representing a sound speed and an attenuation of the ultrasonic wave in the breast 1a are acquired. By collecting the signals while moving up and down the transducer array 3, a three-dimensional image of the subject is acquired.

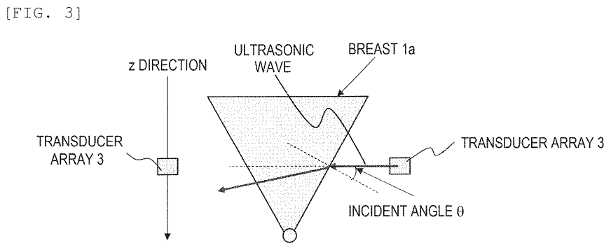

A mechanism unit (not shown) is connected to the transducer array 3. This mechanism unit moves the transducer array 3 up and down in a z direction (up and down direction) as shown in FIG. 3 to emit ultrasonic waves from the transducer array 3 to the breast 1a and receive ultrasonic waves from the breast 1a at each depth in the z direction. Therefore, in the ultrasonic CT apparatus according to the present embodiment, a tomographic image and a transmitted wave image can be acquired at each depth in the z direction.

In the present embodiment, as described later, in order to reduce an incident angle θ of the ultrasonic wave with respect to a normal line to the surface of the breast 1a shown in FIG. 3, the member 20 is pressed against a side surface of the breast 1a to change the inclination. At this time, in the ultrasonic CT apparatus according to the present embodiment, since the transducer array 3 is disposed outside the tank 4 that is filled with water, the member 20 is not interfered with the transducer array 3 even if the member 20 is disposed on an inner wall surface of the tank 4 when performing a breast shaping method in which the member 20 is pressed against the side surface of the breast 1a to change the inclination. Since the transducer array 3 is disposed outside the tank 4, there is a merit that a volume of the tank 4 can be reduced and an amount of water used can be reduced.

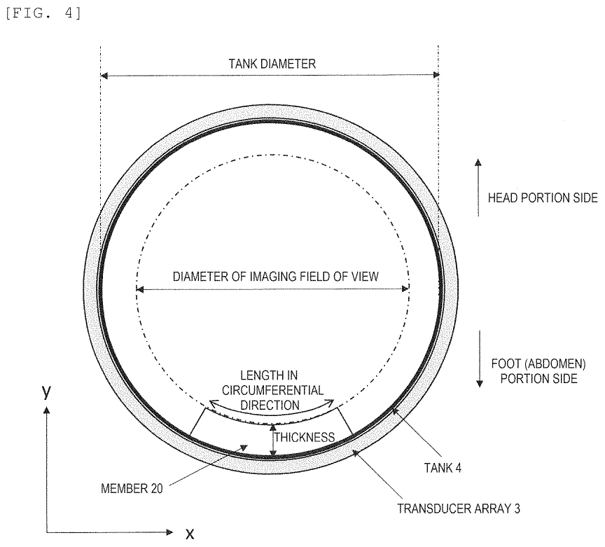

When the tank 4 is viewed from directly above as shown in FIG. 4, the member 20 that transmits ultrasonic waves is disposed on an inner wall of the side surface of the tank 4. The member 20 is a member that contacts the side surface of the breast 1a. In the example of FIG. 4, among side surfaces in the entire circumferential directions of the breast 1a, the member 20 is pressed against aside surface in an angular range on a foot side of the subject 1, and an inclination of a side surface in an angular range on a head portion side is made close to perpendicular.

The member 20 includes any one of a gel, a bag-shaped film into which a liquid is injected, and a tensioned film. As the member 20, it is desirable to use a member having acoustic characteristics such as the sound speed and an attenuation rate close to water. Specifically, for example, the gel used as the member 20 is preferably a material that transmits ultrasonic waves, and examples of the gel include a hydrogel, an acrylamide gel, a gelatin gel, an agarose gel, an oil gel, and a polyvinyl alcohol gel. Besides the gel, a bag filled with degassed water, silicone, polyurethane, and the like may be used.

In the example of FIG. 4, the member 20 is fixed to the inner wall surface of the tank 4. By adopting a structure in which the member 20 is fixed to the inner wall, a soft gel that cannot stand on its own or the bag filled with the degassed water can be used as the member 20.

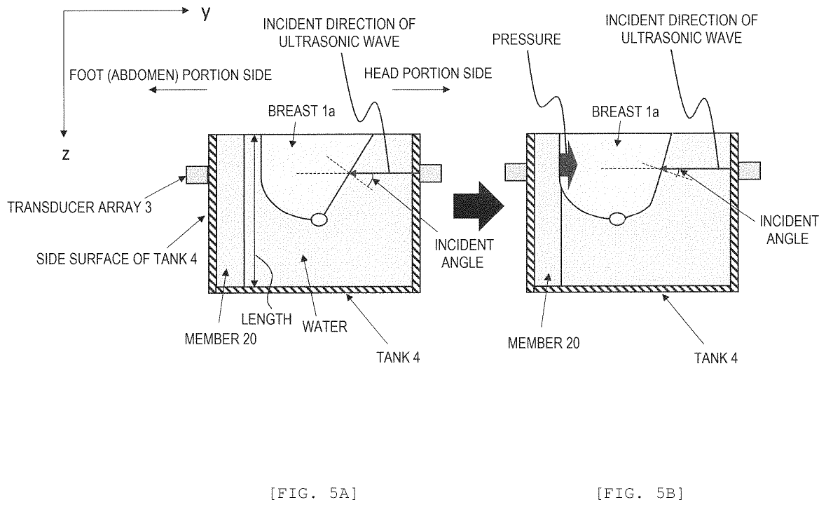

As shown in FIGS. 4 and 5(a), the member 20 is disposed on an inner wall in a predetermined angular range of the side surface on the foot side of the subject 1 among the inner wall of the tank 4 so as to face the side surface on the foot side of the subject 1 among the side surfaces in the entire circumference of the breast 1a of the subject 1.

For example, when the tank 4 has a diameter of 20 cm and an imaging field of view has a diameter of 16 cm, thickness of the member 20 is about 2 cm. A length of the member 20 in a circumferential direction is about 10 cm, which is long enough to contact the breast.

### (Breast Shaping Method)

At the time of imaging, as shown in FIGS. 1 and 5(a), the subject 1 lies on the bed 2 facing downward, inserts the breast into the tank 4 through the through hole 2a, and slightly shifts a body toward the foot side along a body axis, and presses the side surface on the foot side of the subject 1 among side surfaces of the breast 1a against the member 20 as shown in FIG. 5B.

When the subject 1 presses the side surface on the foot side among the side surfaces of the breast 1a against the member 20 of the tank 4, a pressure of a reaction acts on the breast 1a from the member 20, and the breast 1a is deformed. Accordingly, the inclination of the side surface on the head portion side of the subject 1 among the side surfaces of the breast 1a can be made close to an angle close to perpendicular. That is, the inclination of the side surface on the head portion side of the subject 1 having a large inclination in the entire circumference of the breast 1a can be made close to perpendicular by pressing the side surface on the foot side against the member 20. Accordingly, as shown in FIGS. 5(a) and 5(b), it is possible to reduce an incident angle of the ultrasonic wave incident on a surface of the side surface of the breast 1a on the head portion side of the subject 1. The side surface on the foot side of the subject 1 pressed against the member 20 can also be a perpendicular surface along a surface of the member 20.

In a state of FIG. 5B, ultrasonic waves are transmitted from the transducer array 3 for reception. The signal processing unit 8 processes the obtained received signal to generate a reflected image and a transmitted wave image. Accordingly, imaging of the breast can be performed.

Since the breast 1a after shaping in FIG. 5B is deformed so that the inclination of the side surface on the head portion side of the subject 1 among the side surfaces of the breast 1a is made closer to perpendicular than in the state shown in FIG. 5A, an incident direction of the ultrasonic wave can be made close to the normal line of the breast 1a. Therefore, a refraction angle in the z direction when ultrasonic waves are incident on the breast 1a can be reduced, and the reflected waves and transmitted waves that reach the transducer array 3 can be increased. Accordingly, an image quality of the obtained reflection image or transmitted wave image can be improved at low cost.

Among the side surfaces of the breast 1a, the side surface on the foot side of the subject 1 is only pressed against a perpendicular surface of the member 20 by the subject itself, and there is also a merit that a psychological burden on the subject is reduced.

Further, in the configuration of the present embodiment, since the subject 1 presses the breast 1a against the member 20, an effect is obtained that positioning of the breast 1a in the imaging field of view is facilitated.

Although FIGS. 5A and 5B show an example in which a length of the member 20 in the z direction is the same as a depth of the tank 4 in the z direction, the length may be as long as the length of the breast 1a in the z direction, for example, about 10 cm.

Hereinafter, modifications of the first embodiment will be described, but a configuration other than a configuration described in the modifications is similar to that of the first embodiment.

### First Modification of First Embodiment

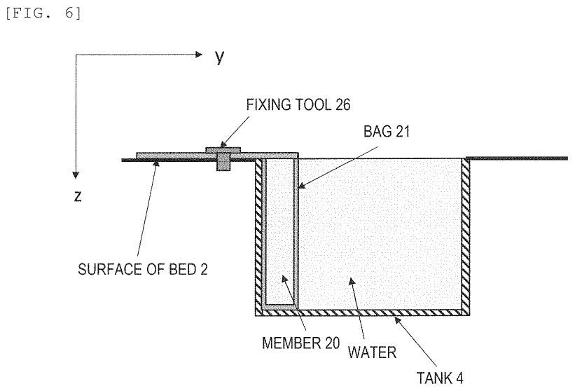

The member 20 such as the gel is configured to be directly fixed to the inner wall of the tank 4 in the first embodiment described above, but the member 20 may be fixed to the inner wall of the tank 4 while being enclosed in a bag 21 as shown in FIG. 6.

In this case, it is desirable to enclose the member 20 so that air is not contained between the bag 21 and the member 20. Thus, by containing the member 20 in the bag 21, it is possible to prevent degradation of the member 20 over time, and it is possible to maintain cleanliness by disinfecting and cleaning a surface of the bag.

In addition, by containing the member 20 in the bag 21, and by fixing an end portion of the bag 21 to a surface of the bed 2 with a fixing tool 26 as shown in FIG. 6, the member 20 can be fixed to the inner wall of the tank 4 in a detachable manner. By fixing the member 20 in the detachable manner, the member 20 can be easily replaced.

A material of the bag 21 is desirably thin enough for transmission of the ultrasonic waves, and is preferably a film made of a polyethylene, a polypropylene, a polyester, a nylon, a polyvinyl chloride, or the like, and having a thickness of 70 μm or less.

### Second Modification of First Embodiment

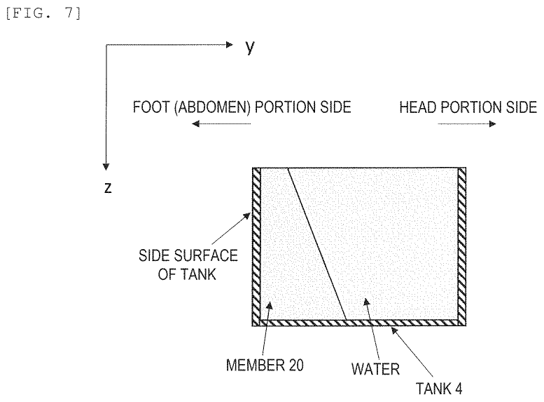

As shown in FIG. 7, the member 20 may have a thickness (y direction) that changes in the z direction and becomes thicker at a deeper position in the z direction. By pressing the breast 1a against the member 20 with such a shape, the breast 1a and the member 20 are brought into closer contact than the configuration shown in FIG. 5A, so that a deformation amount of the breast 1a can be increased. Therefore, among the side surfaces of the breast 1a, the side surface on the head portion side of the subject 1 can be made closer to perpendicular than that in the first embodiment.

### Third Modification of First Embodiment

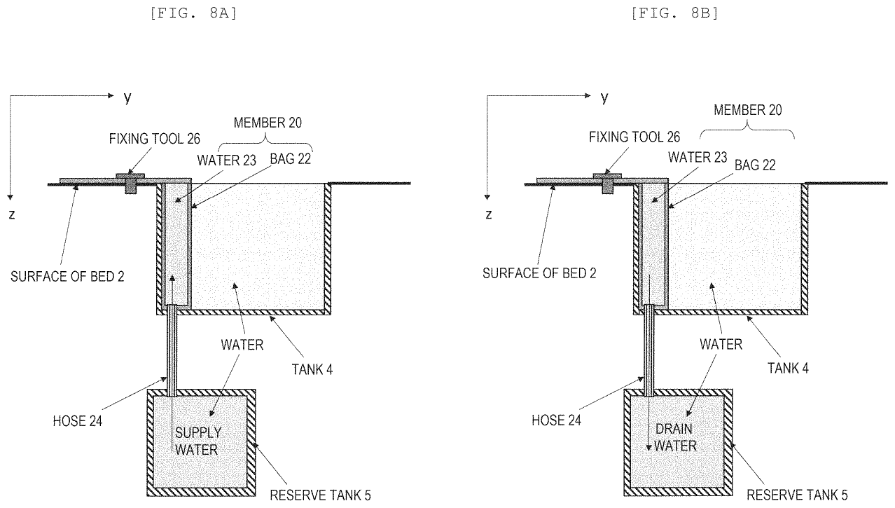

As shown in FIGS. 8A and 8B, when a member with water 23 contained in a bag 22 is used as the member 20, degassed water may be supplied from and drained to the reserve tank 5 to and from the bag 22 through a hose (pipe) 24. With such a configuration, water in the bag 22 can be drained when bubbles are generated, and new degassed water can be supplied to the bag 22, so that the member 20 capable of reducing scattering of ultrasonic waves and propagating can be provided.

An end portion of the bag 22 may be fixed to the bed 2 with the fixing tool 26 similar to that in FIG. 7.

### Fourth Modification of First Embodiment

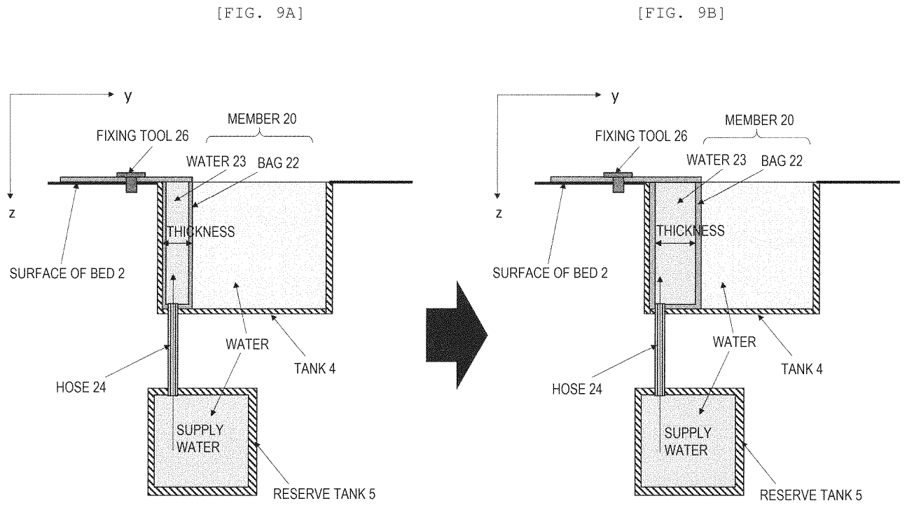

As shown in FIGS. 9A and 9B, when a member with the water 23 contained in the bag 22 is used as the member 20 similar to that in FIGS. 8A and 8B, the bag 22 may be inflated by adjusting an amount of the water 23 supplied to the bag 22 so as to adjust the thickness of the member 20. In this case, a stretchable member is used as the bag 22.

With such a configuration, the thickness of the member 20 can be adjusted according to a size of the breast 1a, and a force with which the member 20 is pressed against the breast 1a can be adjusted.

Alternatively, by increasing the thickness of the member 20, a shape of the breast 1a can be deformed by pressing the breast 1a by the member 20 without pressing the breast 1a against the member 20 by the subject 1 itself.

An amount of water supplied from the reserve tank 5 to the bag 22 may be adjusted by a user manually controlling a pump of the reserve tank 5, or the amount of water supplied from the reserve tank 5 may be controlled by the control unit 7 according to the thickness of the member 20 set by the user or the thickness of the member 20 corresponding to the size of the breast 1a detected by a camera or a sensor, so that the thickness of the member 20 becomes an appropriate thickness corresponding to the size of the breast 1a.

### Fifth Modification of First Embodiment

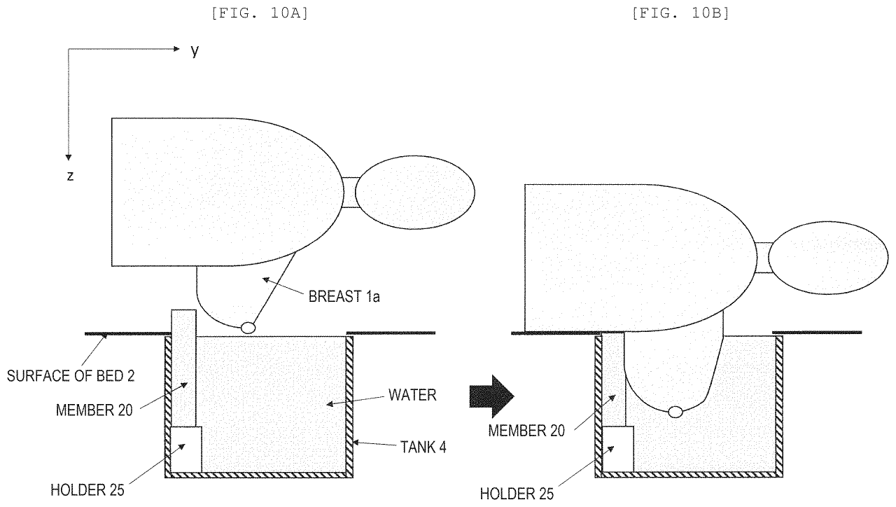

As shown in FIG. 10A, in the configuration of the ultrasonic CT apparatus shown in FIGS. 5A and 5b, the member 20 may be disposed so as to protrude upward (in a negative direction of z) from an upper end of the tank 4. A lower portion of the member 20 is supported by being inserted into a cylindrical holder 25. The lower portion of the member 20 has a size that allows up and down movement (movement in the z direction) within the cylindrical holder 25, and can remain at any position in the holder 25 and stand on its own.

Therefore, as shown in FIG. 10A, the member 20 is disposed to protrude upward (in the negative direction of z) from the upper end of the tank 4, so that the subject 1 can easily grasp a position where the breast 1a is inserted. When the subject 1 inserts the breast 1a into the tank 4, the upper end of the member 20 is pressed by a chest portion and an abdomen portion around the breast 1a of the subject 1 at the same time, and moves in a positive direction of z and stops, so that ultrasonic waves can be transmitted and received similar to that in the first embodiment.

Every time the subject 1 changes, the member 20 is disposed to protrude upward from the upper end of the tank 4 by an operator or the subject 1 itself, or a drive mechanism that holds a lower end of the member 20 and moves the member 20 upward may be disposed in the holder 25.

### Sixth Modification of First Embodiment

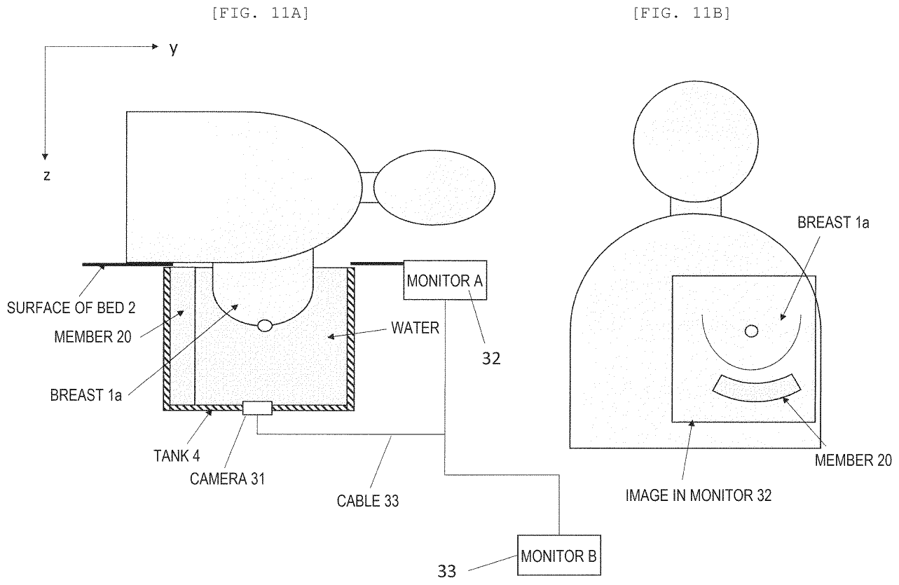

As shown in FIG. 11A, an optical camera 31 maybe installed on a bottom surface of the tank 4, and a positional relationship between the breast 1a and the member 20 may be imaged by the optical camera 31 and output to a monitor (A) 32 and a monitor (B) 33. The monitor (A) is disposed at a position where the subject 1 in the state of facing down on the bed 2 can be seen. The monitor (B) is disposed at a position that can be seen by the operator.

With such a configuration, the subject 1 can grasp the positional relationship between the breast 1a of itself and the member 20 by looking at an image in the monitor (A) 32 as shown in FIG. 11B while the subject 1 is in the state of facing down on the bed 2. Therefore, the subject 1 can change a position thereof while looking at the monitor (A) 32 and press the breast 1a against the member 20 to shape the breast 1a as described in the first embodiment.

The operator of the ultrasonic CT grasps the positional relationship between the breast 1a of the subject 1 and the member 20 by looking at the monitor (B) 33, transmits an instruction to the subject 1, and makes the subject 1 move in accordance with the instruction, so that the positioning the breast 1a can be performed.

### Seventh Modification of First Embodiment

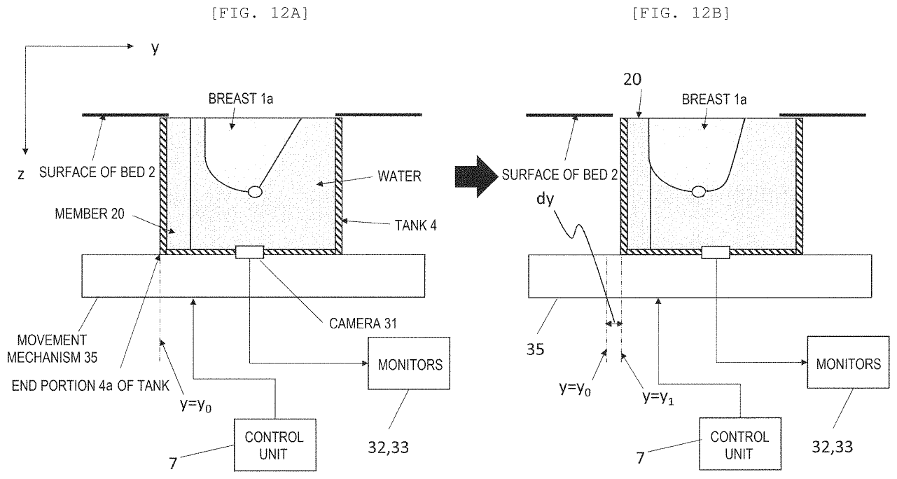

As shown in FIGS. 12(a) and 12(b), in order to move the member 20 in a direction of pressing the member 20 toward the breast 1a, a movement mechanism 35 for moving the tank 4 may be disposed in the ultrasonic CT apparatus. An operation of the movement mechanism 35 is controlled by the control unit 7.

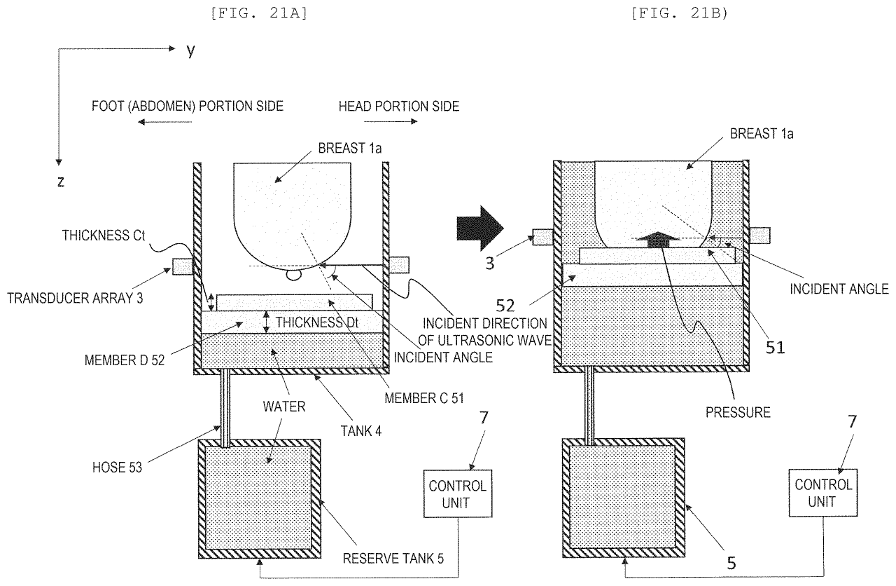

The movement mechanism. 35 includes a mechanism unit that moves the tank 4 in the y direction (a direction parallel to the surface of the bed 2, which is, a horizontal direction) and a drive unit (motor), so that the breast 1a is shaped by pressing the member 20 against the breast 1a. FIG. 21A shows a case where an end portion of the tank 4 is at a position y₀ in the y direction, and FIG. 12B shows a state where the member 20 is pressed against the breast 1a by the movement mechanism 35 moving the tank 4 only by dy in a positive direction of the y direction, such that the end portion of the tank 4 is moved to a position y₁.

The movement of the tank 4 by the movement mechanism 35 is controlled by the control unit 7 controlling the drive unit (motor) of the movement mechanism.

A movement amount of the tank 4 may be determined (adjusted) by the subject 1 or the operator while being confirmed based on an image of the camera 31, that is, the positional relationship between the breast 1a and the member 20 on the monitors 32 and 33, and be instructed to the control unit 7. The control unit 7 may be configured to process the image of the camera 31 to measure a distance between the breast 1a and the member 20, and to move the tank 4 until the member is pressed against the breast 1a. At this time, in consideration of safety, an upper limit may be set for a movable distance of the tank 4. For example, the movable distance of the tank 4 may be 1 cm at the maximum. The tank 4 may be configured to move by a predetermined movement amount.

With such a configuration of the present modification, it is possible to shape the breast 1a by moving the member 20 without the subject 1 moving by itself.

### Eighth Modification of First Embodiment

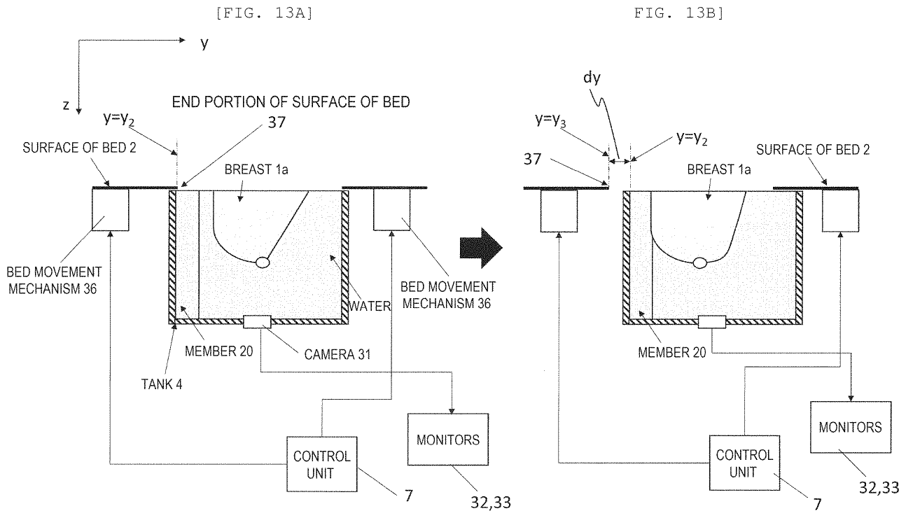

As shown in FIGS. 13A and 13B, a movement mechanism 36 for moving the bed 2 relative to the tank 4 may be disposed in the ultrasonic CT apparatus in order to press the member 20 against the breast 1a.

The movement mechanism 36 includes a mechanism unit that moves the bed 2 in the y direction (the direction parallel to the surface of the bed 2, that is, the horizontal direction) and a drive unit (motor), and by moving the bed 2, the subject 1 is moved in the y direction, and the breast 1a is shaped by pressing the member 20 against the breast 1a.

FIG. 13A shows a case where an end portion 37 of the surface of the bed 2 is at a position y₂ in the y direction, and FIG. 13B shows a state where the breast 1a is pressed against the member 20 by the movement mechanism 36 of the bed 2 moving the bed 2 only by dy in a negative direction of the y direction, and the end portion 37 of the surface of the bed 2 is moved to a position y₃.

Since the control of the movement mechanism 36 over the bed 2 can be performed similar to the control of the movement mechanism 35 according to the sixth modification, the description thereof is omitted here.

### Ninth Modification of First Embodiment

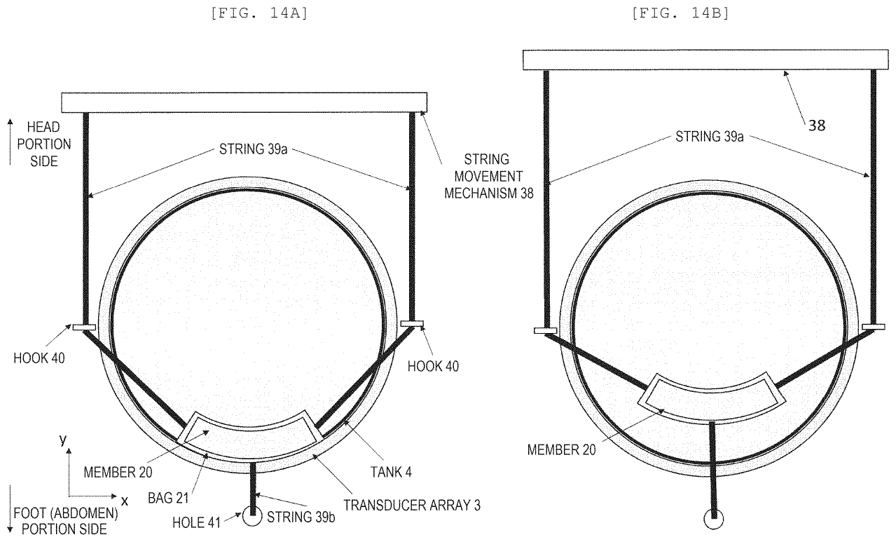

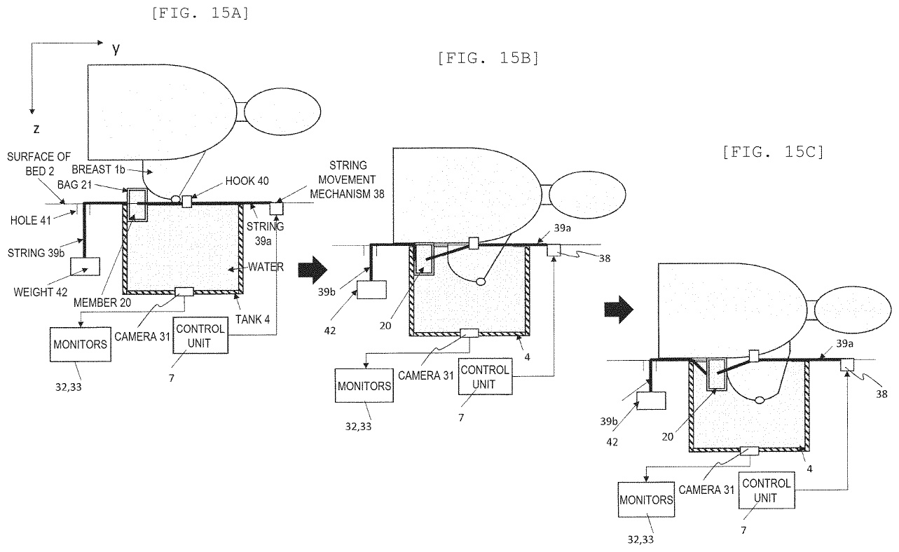

As shown in FIGS. 14A, 14B and FIGS. 15A to 15C, a movement mechanism 38 for moving the member 20 in the tank 4 may be disposed in the ultrasonic CT apparatus as a movement mechanism that relatively moves the member 20 in the direction of pressing the member 20 toward the breast.

Specifically, the movement mechanism 38 is configured to move the member 20 by pulling strings 39a attached to both ends in the circumferential direction of the bag in which the gel 20 is contained or the bag 21 in which the liquid is contained in a head portion direction of the subject 1.

Hooks 40 are fixed to an upper surface of the bed 2, and the strings 39a pass through the hooks 40. A string 39b is attached to a central portion in the circumferential direction of the bag 21 in which the member 20 is contained. The string 39b is pulled in a foot direction of the subject, passes a hole 41 provided on the foot side of the opening 2a of the bed 2 downward from a surface side of the bed 2, and is attached to a weight 42 at a tip end thereof extended downward. Accordingly, forces balanced in three directions from both ends and a center in the circumferential direction is applied to the member 20, and a tension in the circumferential direction and a radial direction of the tank 4 is maintained on the member 20. A balance of forces in three directions can be maintained even when the strings 39a is pulled by the movement mechanism 38 to move the member 20.

As shown in FIGS. 14A and 15A, before the breast 1a is inserted into the tank 4, the member 20 is near a wall surface of the tank 4. At this time, the upper end of the member 20 protrudes upward from the surface of the bed 2.

Next, as shown in FIG. 15B, when the subject 1 faces down on the bed 2 and the breast 1a is inserted into the tank 4 through the opening 2a, the upper end of the member 20 is pressed downward by the chest portion or the abdomen portion around the breast 1a of the subject 1, and moves in the positive direction of z.

Finally, as shown in FIG. 15C, the member 20 in is moved the tank 4 by pulling the strings 39a in the positive direction of y (a head direction of the subject 1) by the movement mechanism 38 with the string 39b, and the member 20 is pressed against the breast 1a. A movement amount of the member 20 may be adjusted by the subject 1 or the operator while being confirmed based on the image of the camera 31, that is, the positional relationship between the breast 1a and the member 20 on the monitors 32 and 33.

At this time, in consideration of safety, an upper limit may be set for a movable distance of the member 20. For example, the movable distance of the member 20 may be 1 cm at the maximum.

As described above, according to such a configuration of the present modification, it is possible to shape the breast 1a by moving the member 20 in the tank 4 without the subject 1 moving by itself.

### Tenth Modification of First Embodiment

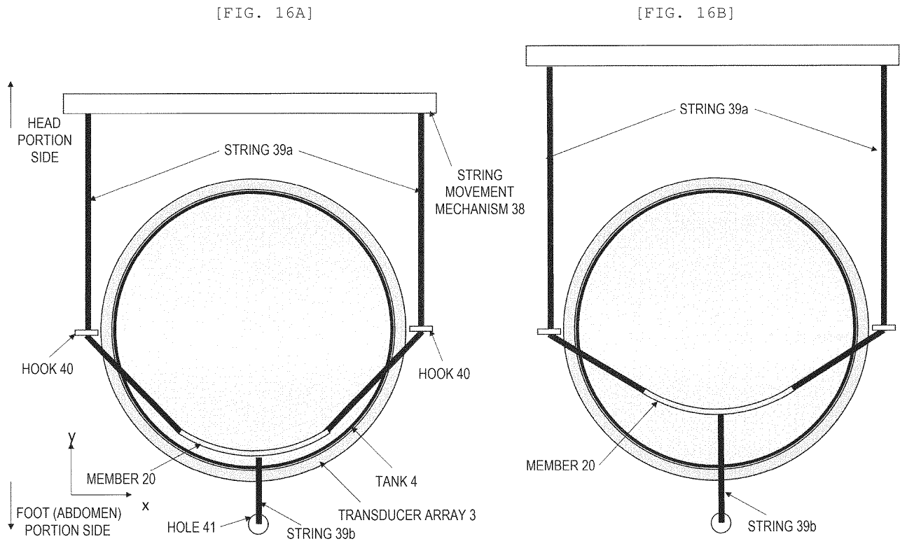

In the ninth modification, a thin film can be used as the member 20 as shown in FIG. 16A and 16B.

Examples of a film material include the polyethylene, the polypropylene, the polyester, the nylon, and the polyvinyl chloride.

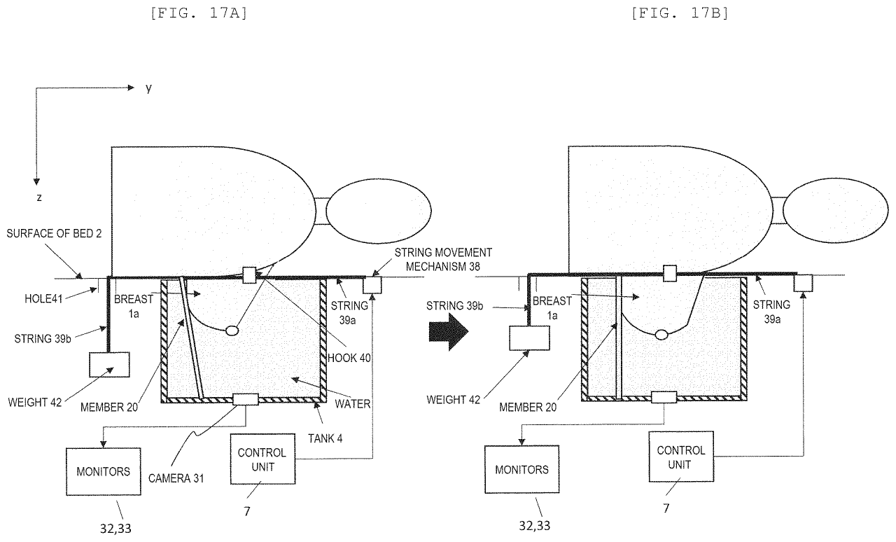

As shown in FIGS. 17A and 17B, a lower end of the film may be fixed to a bottom portion of the tank 4.

### Eleventh Modification of First Embodiment

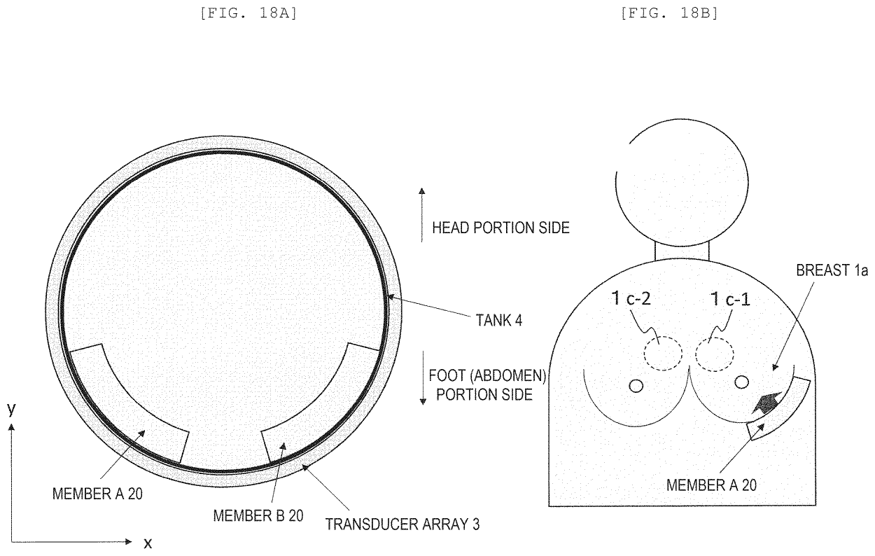

As shown in FIG. 18A, a plurality of members 20 according to the first embodiment may be disposed in the tank 4, and positions for disposing the members 20 may be different from those in FIG. 4.

As shown in FIG. 18B, an inclination angle of the side surface of the human breast 1a is the largest in areas 1c-1 and 1c-2 on the head portion side among the side surfaces in the entire circumference of the breast 1a and close to the body axis of the subject 1. Therefore, in both the left and right breasts 1a, pressing the members 20 in areas facing each other and sandwiching nipples with respect to the areas 1c-1 and 1c-2 having large inclination angles, specifically, areas close to an outside of the body axis of the subject 1 on the side surface the foot side of the breast 1a is suitable for shaping the inclination of the breast 1a.

Therefore, in the present modification, among inner walls of the tank 4, a member (A) 20 and a member (B) 20 are disposed in symmetrical areas on an inner wall of the foot side of the subject 1 and sandwiching an axis connecting the head portion side and a foot portion side of the subject 1 through the center of the tank 4. Accordingly, when the breast 1a on a left side is inserted into the tank 4, by pressing against the member (A), the breast 1a can be shaped so as to effectively reduce the inclination of the area 1c-1 having the large inclination. On the other hand, when the breast 1a on a right side of the subject 1 is inserted into the tank 4, by pressing against the member (B), the breast 1a can be shaped so as to effectively reduce the inclination of the area 1c-2 having the large inclination.

### Twelfth Modification of First Embodiment

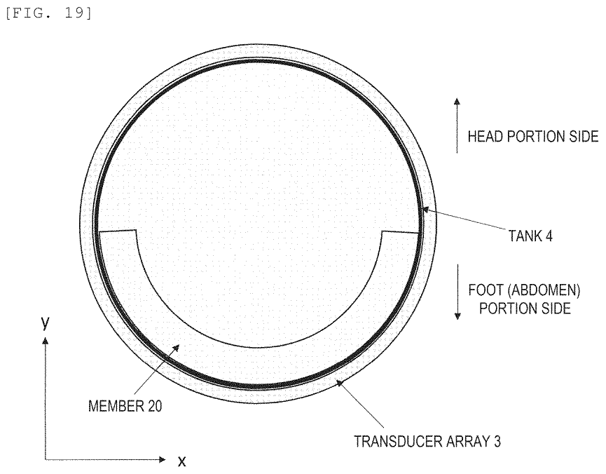

Although the configuration in which the plurality of members 20 are disposed in the tank 4 has been described in the eleventh modification, as shown in FIG. 19, the member 20 may be disposed on an entire inner wall of a half circumference in the circumferential direction of the tank 4. However, an inner wall on which the member 20 is disposed is an inner wall of the tank 4 on the foot side of the subject 1.

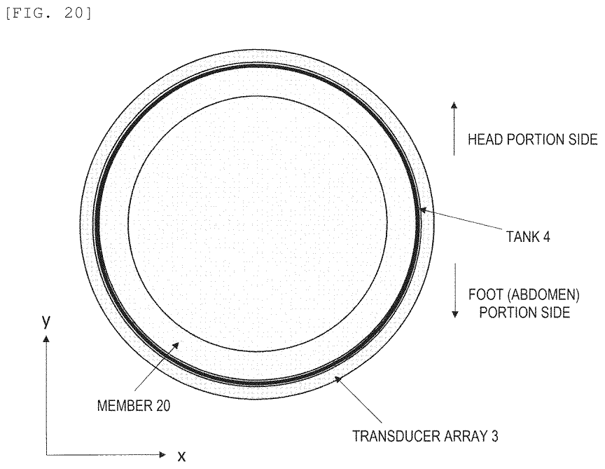

Alternatively, as shown in FIG. 20, the member 20 may be disposed in an entire area in the circumferential direction of the tank 4.

When the member 20 is disposed as shown in FIGS. 19 and 20, the subject 1 can freely select a direction in which the breast 1a is pressed.

### Second Embodiment

Although the breast 1a is deformed by pressing the side surface of the breast 1a against the member 20 according to the first embodiment, in an ultrasonic CT apparatus according to a second embodiment, the breast 1a is deformed by pushing up the breast 1a from a nipple side to a chest wall direction with a plate-shaped member 51, so that the inclination of the side surface of the breast 1a in the tank 4 is made close to perpendicular, and the incident angle of the ultrasonic wave is reduced.

Specifically, as shown in FIGS. 21A and 21B, the plate-shaped member (C) 51 and a plate-shaped member (D) 52 are disposed in the tank 4 in parallel to the bottom surface of the tank 4. The member (D) 52 is made of a material that floats in water. The member (C) 51 is disposed on the member (D) and floats due to buoyancy of the member (D) 52.

Therefore, in response to a command from the control unit 7, water is supplied from the reserve tank 5 to the tank 4 from the bottom surface thereof through the hose 53, so that the member (C) 51 and the member (D) 52 move upward in the negative direction of z, and finally, the member (C) 51 can be brought into close contact with the breast 1a by being pushed up by the buoyancy of the member (D), and the breast 1a can be deformed due to pressure applied by the buoyancy to the breast 1a. Accordingly, the side surface of the breast 1a can be made close to perpendicular, and the incident angle of the ultrasonic wave from the transducer array 3 can be reduced.

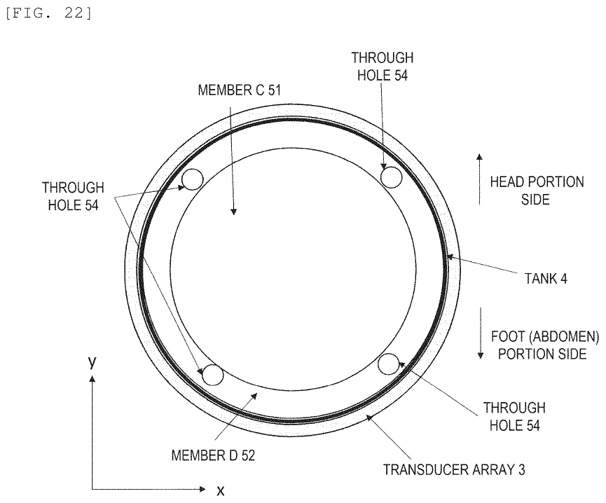

FIG. 22 shows shapes of the member (C) 51 and the member (D) 52 on an xy plane. The member (C) 51 and the member (D) 52 are circular, and diameters of the member (C) 51 and the member (D) 52 are smaller than a diameter of the inner wall of the tank 4. For example, when the tank 4 has a diameter of 200 mm in the inner wall, the diameters of the members (C) 51 and (D) 52 are about 160 mm and 198 mm, respectively. In the member (D), a plurality of through holes 54 are provided in a peripheral edge portion as water passages.

Since thickness of an ultrasonic beam from the transducer array 3 is finite, the member (C) 51 in close contact with the breast 1a may be reflected in the imaged tomographic image. Therefore, a material of the member C51 is preferably a material that transmits ultrasonic waves, and similar to the case of the first embodiment, the hydrogel, the acrylamide gel, the gelatin gel, the agarose gel, the oil gel, the polyvinyl alcohol gel, or the like can be used. Besides the gel, a bag filled with degassed water, silicone, polyurethane, and the like may be used as the member (C) 51. A member such as the gel, silicon, or polyurethane may be contained in the bag.

A material of the member (D) 52 is preferably a material having a density lower than that of water, and a foamed polystyrene, a high-density polyethylene, a foamed polyethylene, an ethylene vinyl acetate copolymer, a bag containing air, or the like can be used.

Thickness Ct of the member (C) 51 is set to, for example, about 1 cm so that the member (D) 52 is not reflected in the tomographic image. A value of the thickness Ct is not limited to 1 cm, but depends on the thickness of the ultrasonic beam, and as the thickness of the beam is reduced, the value of the thickness Ct can be reduced. When the thickness of the ultrasonic beam is large, the reflection of the member (D) 52 on the tomographic image can be eliminated by increasing the thickness Ct.

The thickness Dt of the member (D) 52 is a parameter for adjusting the buoyancy (pressure applied to the breast 1a), and the buoyancy increases as the thickness increases. As an example, the thickness is about 3 cm. However, a value of the thickness Dt is not limited to 3 cm. When it is desired to increase the pressure applied to the breast 1a, the thickness Dt may be set to 3 cm or more, and when it is desired to reduce the pressure, Dt may be set to 3 cm or less. Alternatively, it is also possible to adjust the pressure by using a member (D) 52 with different thickness repeatedly.

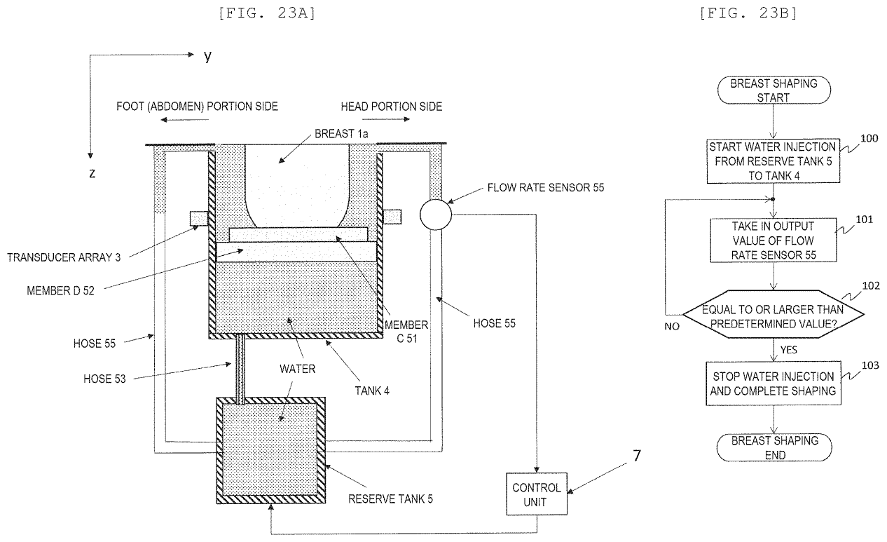

As shown in FIG. 23A, it is desirable that a hose 55 for returning water overflowing from the tank 4 to the reserve tank 5 is provided near an opening of the tank 4. In this case, with a flow rate sensor 55 disposed in an intermediate position of the hose 55, and by taking in an output of the flow rate sensor 55, the control unit 7 can automatically perform shaping the breast 1a.

Specifically, for example, when an operation of the control unit 7 is implemented by software, the operation is as shown in the flowchart of FIG. 23B. First, when the control unit 7 receives an instruction to start breast shaping from the operator or the subject 1, the control unit 7 operates the pump of the reserve tank 5 to start water injection from the reserve tank 5 to the tank 4 (step 100). Accordingly, water is injected into the tank 4 from the bottom surface to which the hose 53 is connected, and a water level gradually rises. Along with this, the member (D) 52 and the member (C) 51 mounted on the member (D) 52 gradually float and are pushed up to be in contact with the nipple of the breast 1a, and the member (C) 51 is pressed against the breast 1a with a force that tends to lift due to the buoyancy of the member (D) 52. When water passes through the through hole 54 in the peripheral edge portion of the member (D) 52, fills an upper portion of the member (D) 52, and reaches the hose 55 near the opening of the tank 4, the water overflows from the tank 4, and returns to the reserve tank 5 through the hose 55.

The flow rate sensor 55 detects a flow rate of water flowing through the hose 55. The control unit 7 takes in the flow rate output from the flow rate sensor 55 (step 101), and when the flow rate reaches a predetermined value or more, the control unit 7 stops the water injection from the reserve tank 5 to the tank 4 (steps 102 and 103).

Accordingly, the subject 1 or the operator only instructs the start of shaping the breast 1a, and under the control of the control unit 7, a push-up force by the buoyancy of the member (D) 52 can be applied to the breast 1a to deform the breast 1a and make the side surface close to perpendicular. Therefore, the ultrasonic CT apparatus can acquire a reflected wave image and a transmitted wave image after emitting ultrasonic waves by the transducer array 3.

### First Modification of Second Embodiment

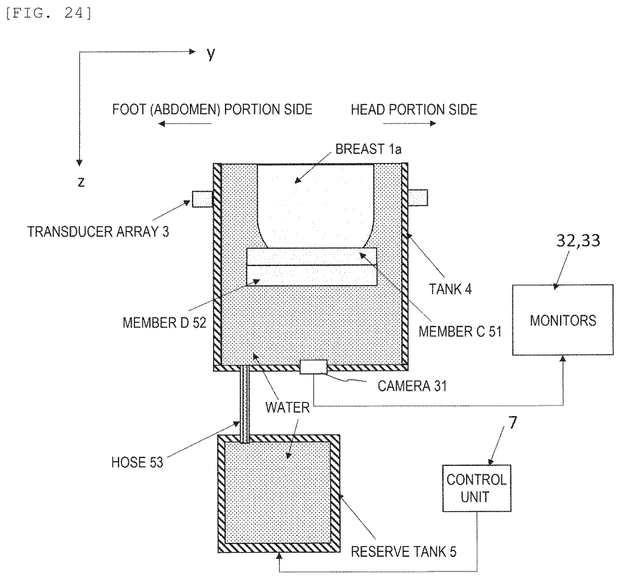

In the second embodiment described above, water overflowing from the tank 4 is detected by the flow rate sensor 55, but as shown in FIG. 24, the camera 31 may be installed on the bottom portion of the tank 4, so as to detect the water overflowing from the tank 4 by an image of the camera 31.

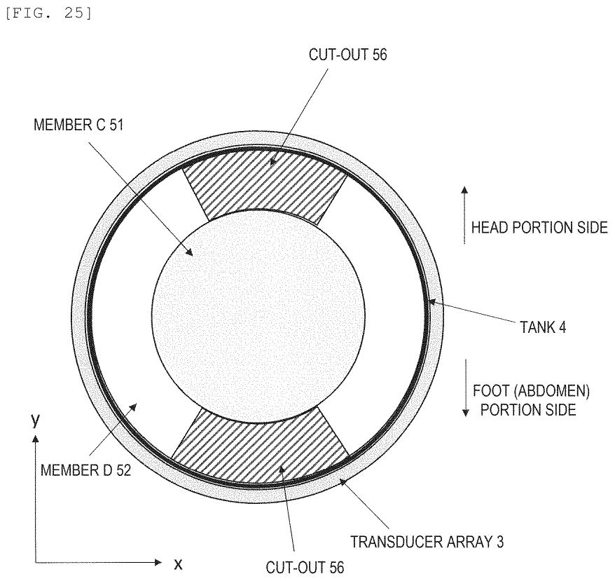

At this time, as shown in FIG. 25, a cut-out 56 is provided on the member (D) 52 so that the camera 31 can image a position of a water surface. A shape of the cut-out 56 may be any shape as long as the camera 31 can image the water surface, but may be, for example, a fan-shaped cut-out as indicated by oblique lines in FIG. 25.

The diameter of the member (C) 51 may be smaller than that shown in FIG. 22 of the second embodiment, and may be, for example, about 140 mm.

For example, when the subject 1 or the operator confirms on the monitors 32 and 33 that water reaches an upper surface of the tank 4 with an image of the camera 31, the subject 1 or the operator instructs the control unit 7 to stop the water supply from the input and output unit 10.

In addition, the control unit 7 may detect that the water reaches the upper surface of the tank 4 by processing the image imaged by the camera 31, and stop the water supply to the reserve tank 5.

### Second Modification of Second Embodiment

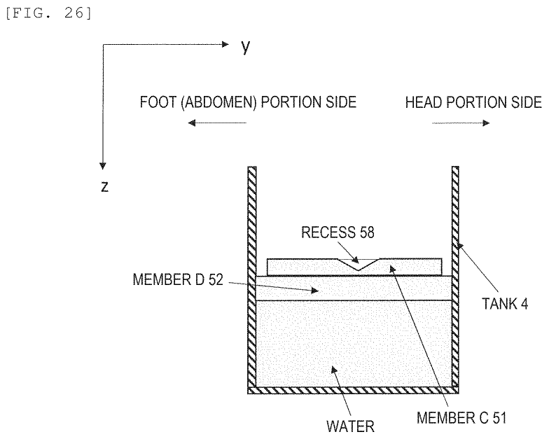

As shown in FIG. 26, the member (C) 51 may have a recess 58 with a size big enough to contain the nipple of the breast 1a. With such a shape, the member (C) 51 can be brought into closer contact with the breast 1a.

### Third Modification of Second Embodiment

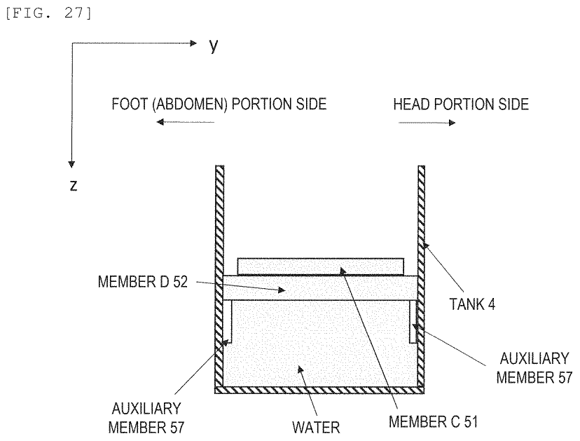

As shown in FIG. 27, an auxiliary member 57 maybe coupled to a lower surface of the peripheral edge portion of the member (D) 52. The auxiliary member 57 has a shape in which an outer peripheral surface is along the inner wall surface of the tank 4, and acts as a guide along the inner wall surface of the tank 4 when the member (D) 52 moves upward (in the negative direction of z) along with the water injection into the tank 4. Accordingly, shakiness when the member (D) 52 rises can be reduced, and the member (D) 52 can rise while maintaining in a horizontal state with respect to the xy plane.

The auxiliary member 57 maybe formed of the same material as the member (D) 52, or may be made of a resin such a light plastic and a hard plastic.

The auxiliary member 57 may be disposed on the entire circumference of the member (D) 52, or may be disposed only in several places.

### Fourth Modification of Second Embodiment

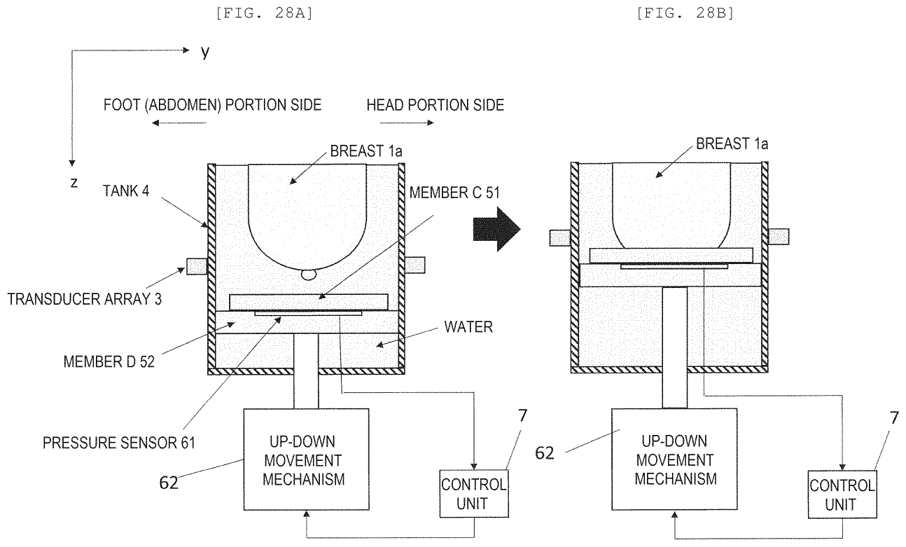

As shown in FIGS. 28A and 28B, as a method of pushing up the breast 1a from the nipple to the chest wall direction, a mechanism 62 that moves up and down by a motor may be used instead of buoyancy.

When the up-down movement mechanism 62 is used, it is desirable to detect a pressure to push up the breast 1a and control the up-down movement mechanism 62. Therefore, in the configuration shown in FIGS. 28A and 28B, a pressure sensor 61 is disposed between the member (C) and the member (D).

At the time of breast shaping, the control unit 7 operates a drive unit (for example, a motor) of the up-down movement mechanism 62 to raise the member (C) 51 and the member (D) 52 and monitors an output of the pressure sensor 61. When the output of the pressure sensor 61 reaches a reference pressure after the member (C) 51 is brought into close contact with the breast 1a, the control unit 7 stops the drive unit of the up-down movement mechanism 62.

According to the present modification, the breast 1a can be pushed up with a predetermined pressure without using buoyancy, and the side surface can be made close to perpendicular. Therefore, the material of the member (D) 52 may not be a material that floats in water, and for example, a metal such as stainless steel or a resin such as plastic can be used.

### Fifth Modification of Second Embodiment

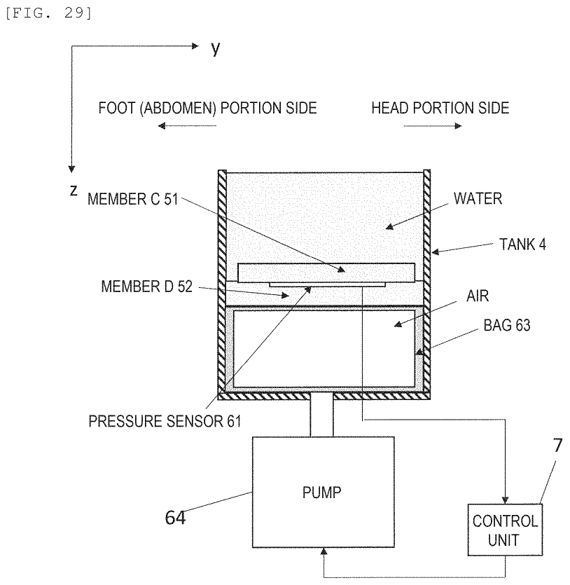

As shown in FIG. 29, as a mechanism for pushing up the breast 1a from the nipple to the chest wall direction, a mechanism including a bag 63 containing air and a pump 64 that sends air to the bag 63 to expand and contract the bag 63 may be used. When the control unit 7 controls an amount of air supplied from the pump 64 to the bag 63, an amount by which the bag 63 is inflated is adjusted, and a push-up amount of the breast 1a is adjusted.

The pressure sensor 61 is installed between the member (C) 51 and the member (D) 52 similar to that in the fourth modification of the second embodiment. The control unit 7 takes in the output of the pressure sensor 61, and stops an inflow of air into the bag 63 by the pump 64 when the member (C) 51 reaches a certain reference pressure after being brought into close contact with the breast 1a.

### Sixth Modification of Second Embodiment

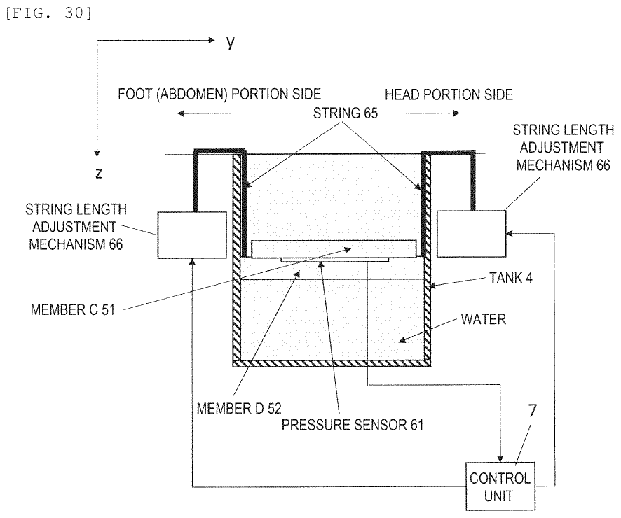

As shown in FIG. 30, as a mechanism for pushing up the breast 1a from the nipple to the chest wall direction, a mechanism for moving strings 65 attached to an end portion of the member (D) 52 up and down may be used. For example, the strings 65 are attached to two end portions of the member (D) 52, and an adjustment mechanism 66 that pulls a tip end of the string 65 out of the opening of the tank 4 and winds the string 65 is attached. Accordingly, by winding the string 65 by the adjustment mechanism 66, the string 65 can be moved up and down in the tank 4.

The pressure sensor 61 is installed between the member (C) 51 and the member (D) 52 similar to that in the fourth modification of the second embodiment. A control operation of the control unit 7 is also similar to that of the fourth modification of the second embodiment.

### Third Embodiment

In the third embodiment, an ultrasonic CT apparatus that combines the configurations of the first and second embodiments will be described.

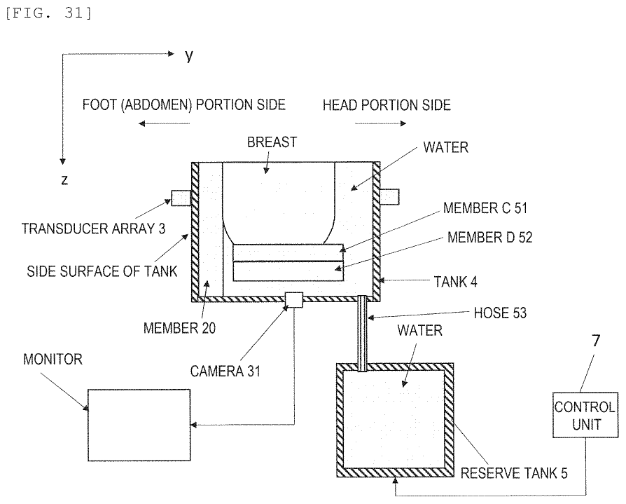

As shown in FIG. 31, the breast 1a is deformed by pressing the side surface of the breast 1a against the member 20 disposed on a side surface of the inner wall of the tank 4, and by being pushed up by the member (C) 51 on the member (D) 52 from the nipple to the chest wall direction of the breast 1a.

Here, as a mechanism for raising the member (D) 52 and the member (C) 51, a structure shown in FIGS. 24 and 25 is adopted, and the buoyancy of the member (D) is used. To stop the water injection into the tank 4, the operator instructs the control unit 7 to stop the water supply while looking at the image imaged by the camera 31 on the monitor 33.

As a structure for pressing the member 20 against the side surface of the breast 1a, the structure of FIGS. 11A and 11B are used. The operator grasps the positional relationship between the breast 1a of the subject 1 and the member 20 while looking at the monitor 33 and instructs the subject 1 to move, and the subject 1 presses the breast 1a thereof to the member 20.

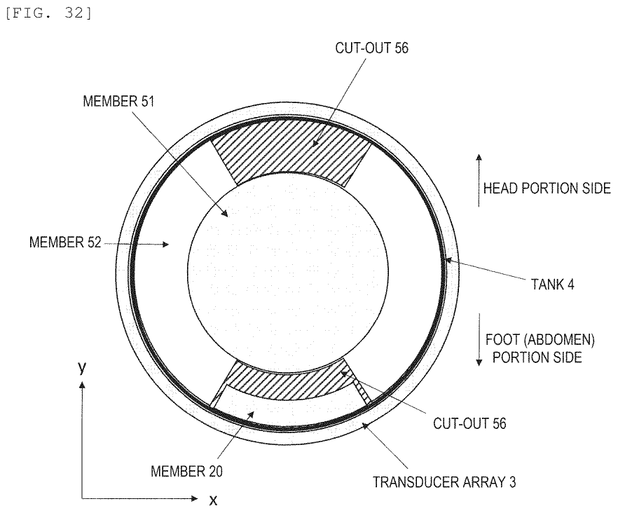

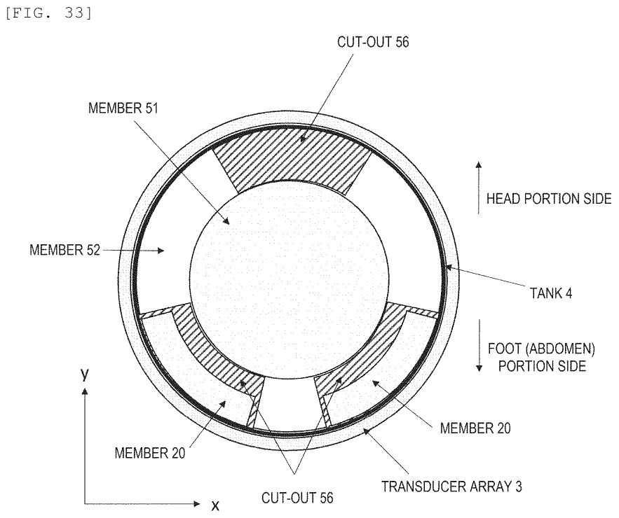

As shown in FIG. 32 or FIG. 33, the member (D) 52 is disposed so that the member 20 is positioned in the cut-out 56 of the member (D) 52. Accordingly, the member (D) 52 can move up and down without interfering with the member 20. As shown in FIG. 33, the number of the members 20 may be plural. In that case, cut-outs of the member 52 are provided in accordance with positions of the plurality of the members 20.

Instead of the operator, the subject 1 may move to look at the monitor 32 by itself and press the member 20 against the breast 1a.

Various members described in the first embodiment and the modifications of the first embodiment can be used as the member 20.

As a mechanism for pressing the member 20 against the breast 1a, it is certainly possible to adopt various modifications of the first embodiment such as moving the tank 4 as shown in FIGS. 12A and 12B, moving the bed as shown in FIGS. 13A and 13B, and moving the member 20 with the strings as shown in FIGS. 14A and 14B.

As a structure for moving the members 51 and 52, it is possible to adopt various modifications of the second embodiment, such as the structure using the motor as shown in FIGS. 28A and 28B, the structure in which the bag is inflated with air as shown in FIG. 29, and the structure in which the strings are pulled up as shown in FIG. 30, in addition to the structure using buoyancy. The member 20 may be fixed to the bed in a state of being contained in the bag, as in the modification of the first embodiment (FIG. 6).

### REFERENCE SIGN LIST

1: subject
1a: breast
2: bed
3: transducer array
4: tank
5: reserve tank
7: control unit
8: signal processing unit
9: storage unit
10: input and output unit
20: member
21: bag
22: bag
23: water
24: hose
25: holder
26: fixing tool
31: camera
32, 33: monitor
36: movement mechanism of bed
37: end portion of surface of bed
38: movement mechanism of string
39a, 39b: string
40: hook
41: hole
42: weight
51: member
52: member
53: hose
54: through hole
55: hose
56: cut-out
57: auxiliary member
58: recess
61: pressure sensor
62: up and down movement mechanism
63: bag
64: pump
64: string
66: adjustment mechanism of string length

1. An ultrasonic CT apparatus, comprising:
a bed provided with a through hole to which a breast of a subject is inserted;
a container disposed below the through hole and provided with an opening to which the breast is inserted; and
a transducer array configured to emit an ultrasonic wave around the breast in the container, and to receive an ultrasonic wave from the breast, wherein
in the container, a member that propagates or transmits the ultrasonic wave is disposed, and the member is configured to be relatively pressed against a side surface or a nipple portion of the breast such that an inclination of the side surface of the breast is close to perpendicular with respect to a chest wall surface.

2. The ultrasonic CT apparatus according to claim 1, wherein the member is a member to be brought into contact with the side surface of the breast.

3. The ultrasonic CT apparatus according to claim 2, wherein among side surfaces of the breast, the member is pressed against a side surface on a foot side of the subject, such that an inclination of a side surface on a head portion side is close to perpendicular.

4. The ultrasonic CT apparatus according to claim 2, wherein the member includes any one of a gel, a bag-shaped film into which a liquid is injected, and a tensioned film.

5. The ultrasonic CT apparatus according to claim 2, wherein the member is fixed to an inner wall surface of the container.

6. The ultrasonic CT apparatus according to claim 2, further comprising:
a movement mechanism configured to relatively move the member in a direction of pressing the member toward the breast.

7. The ultrasonic CT apparatus according to claim 6, wherein the movement mechanism is a mechanism configured to move the member in the container.

8. The ultrasonic CT apparatus according to claim 6, wherein the movement mechanism is a mechanism configured to move the container provided with the member relative to the breast.

9. The ultrasonic CT apparatus according to claim 4, wherein the member is the bag-shaped film, and is connected to a pump configured to inject the liquid into the bag-shaped film at a predetermined pressure.

10. The ultrasonic CT apparatus according to claim 4, wherein the member includes the film and a string that applies tension by pulling the film.

11. The ultrasonic CT apparatus according to claim 2, wherein the member is disposed only in an area among inner walls of side surfaces of the container that contacts an angular range of a part of the side surfaces around the breast.

12. The ultrasonic CT apparatus according to claim 11, wherein the angular range of the part of the side surfaces around the breast is a range of a side surface of the breast on a foot side of the subject.

13. The ultrasonic CT apparatus according to claim 1, wherein the member is a plate-shaped member configured to be brought into contact with a nipple side of the breast such that the breast is pushed up toward a chest wall of the subject.

14. The ultrasonic CT apparatus according to claim 13, further comprising:
a buoyancy member on which the plate-like member is mounted and having buoyancy in water filled in the container to push up the plate-like member.

15. The ultrasonic CT apparatus according to claim 13, further comprising:
a push-up mechanism configured to move the plate-shaped member to a chest wall direction of the subject.

16. The ultrasonic CT apparatus according to claim 14, further comprising:
a side member fixed to the inner wall surface of the container and to be pressed against the side surface of the breast, wherein
the buoyancy member is provided with a cut-out at a position where the side member is disposed.

17. The ultrasonic CT apparatus according to claim 1, wherein the transducer array is disposed outside the container.

18. The ultrasonic CT apparatus according to claim 1, wherein a camera configured to image a positional relationship between the breast and the member is disposed at a bottom portion of the container.

19. A container for ultrasonic CT apparatus which is attached to a lower portion of a breast insertion through hole of a bed of an ultrasonic CT apparatus, and to which the breast is inserted, wherein
a member that propagates or transmits an ultrasonic wave is disposed, and the member is configured to be pressed against a side surface or a nipple portion of the breast such that an inclination of the side surface of the breast is close to perpendicular.

20. A breast imaging method comprising:
a breast shaping step of relatively pressing a member that propagates or transmits an ultrasonic wave against a side surface or a nipple portion of a breast of a subject which is inserted, from a through hole of a bed, into a container disposed below the through hole, such that an inclination of the side surface of the breast is close to perpendicular; and
an imaging step of generating an image from a received signal, the received signal being obtained by emitting an ultrasonic wave around the breast after shaping, and receiving a reflected wave and/or a transmitted wave.
